(19) United States
(12) Patent Application Publication
(10) Pub. No.: US 2024/0216275 A1
(43) Pub. Date: Jul. 4, 2024

(54) METERED DOSE INHALERS AND SUSPENSION COMPOSITIONS

(71) Applicant: KINDEVA DRUG DELIVERY L.P., Woodbury, MN (US)

(72) Inventors: Philip COCKS, West Bridgford (GB); Alexander SLOWEY, Stapleford (GB); Benjamin MYATT, Leicester (GB); Sarah WRIGGLESWORTH, Loughborough (GB); James LISTER, Nottinghamshire (GB)

(21) Appl. No.: 18/599,422

(22) Filed: Mar. 8, 2024

Related U.S. Application Data
(63) Continuation of application No. PCT/US2022/042958, filed on Sep. 8, 2022.
(60) Provisional application No. 63/241,677, filed on Sep. 8, 2021.
(60) Provisional application No. 63/315,337, filed on Mar. 1, 2022.
(60) Provisional application No. 63/328,120, filed on Apr. 6, 2022.

(51) Int. Cl. A61M 15/00; A61K 31/165; A61K 31/57; A61K 31/58; A61K 47/02; A61K 47/10

(52) U.S. Cl. CPC A61M 15/0065; A61K 31/165; A61K 31/57; A61K 31/58; A61K 47/02; A61K 47/10; A61M 15/0001

(57) ABSTRACT

Various embodiments of a metered dose inhaler are disclosed. The inhaler includes a metering valve, a canister, and an actuator having an actuator nozzle. The canister includes a formulation having greater than 70% by weight of HFO-1234ze(E), and at least one active pharmaceutical ingredient suspended in the formulation to form a suspension.

The present application claims priority to U.S. Provisional Application No. 63/241,677, filed Sep. 8, 2021, U.S. Provisional Application No. 63/315,337, filed Mar. 1, 2022, and U.S. Provisional Application No. 63/328,120, filed Apr. 6, 2022, all of which are incorporated herein by reference in their entirety.

### BACKGROUND

Delivery of aerosolized medicament to the respiratory tract for the treatment of respiratory and other diseases can be done using, by way of example, pressurized metered dose inhalers (pMDI), dry powder inhalers (DPI), or nebulizers. pMDIs are familiar to many patients who suffer from asthma or chronic obstructive pulmonary disease (COPD). pMDI devices can include an aluminum canister, sealed with a metering valve, that contains medicament formulation. Generally, a typical current medicament formulation includes one or more medicinal compounds present in a liquefied hydrofluoroalkane (HFA) propellant.

Historically, the propellants in most pMDIs have been chlorofluorocarbons (CFCs). However, environmental concerns during the 1990s led to the replacement of CFCs with hydrofluoroalkanes (HFAs) as the most commonly used propellant in pMDIs. Although HFAs do not cause ozone depletion, they do have a stated high global warming potential (GWP), which is a measurement of the future radiative effect of an emission of a substance relative to that of the same amount of carbon dioxide (CO₂). The two HFA propellants most commonly used in pMDIs are HFA-134a (CF₃CH₂F) and HFA-227 (CF₃CHFCHF₃) having stated 100-year GWP values of 1300 to 1430 and 3220 to 3350, respectively.

Various other propellants have been proposed over the years. Among them, hydrofluoroolefins (HFOs) and carbon dioxide (CO₂) have been mentioned as a potential propellant for pMDIs, but no pMDI product has been successfully developed or commercialized using either as a propellant.

### SUMMARY

It has now been found that despite HFO-1234ze(E)'s differences from other pMDI propellants, a practical pMDI can be made using HFO-1234ze(E). One advantage of such pMDIs is HFO-1234ze(E)'s stated GWP of less than 1.

In one embodiment, a pMDI (also referred to herein as an MDI or metered dose inhaler) is provided that includes: a metering valve: a canister; and an actuator that includes an actuator nozzle; wherein the canister includes a formulation (i.e., composition), the formulation including greater than 70% by weight of propellant HFO-1234ze(E), ethanol, and at least one active pharmaceutical ingredient (API) suspended in the formulation to form a suspension. In certain embodiments, the formulation further includes ethanol. In certain embodiments, the API is selected from beta agonists (short- or long-acting beta agonists), corticosteroids, anticholinergic agents, TYK inhibitors, and combinations thereof.

In one embodiment, a metered dose inhaler is provided that includes: a metering valve: a canister: and an actuator that includes an actuator nozzle: wherein the canister includes a formulation, the formulation including a propellant including HFO-1234ze(E), ethanol, and an active pharmaceutical ingredient including fluticasone or a pharmaceutically acceptable salt or ester thereof (e.g., fluticasone propionate), wherein the fluticasone or pharmaceutically acceptable salt or ester thereof is suspended in the formulation to form a suspension.

In one embodiment, a metered dose inhaler is provided that includes: a metering valve: a canister: and an actuator that includes an actuator nozzle: wherein the canister includes a formulation, the formulation including a propellant including HFO-1234ze(E), ethanol, and an active pharmaceutical ingredient including salbutamol (i.e., albuterol) or a pharmaceutically acceptable salt or ester thereof (i.e., salbutamol sulfate), wherein the salbutamol or pharmaceutically acceptable salt or ester thereof is suspended in the formulation to form a suspension.

In one embodiment, a metered dose inhaler is provided that includes: a metering valve: a canister; and an actuator that includes an actuator nozzle; wherein the canister includes a formulation, the formulation including a propellant including HFO-1234ze(E), ethanol, and an active pharmaceutical ingredient including mometasone or a pharmaceutically acceptable salt or ester thereof (e.g., mometasone furoate), wherein the mometasone or pharmaceutically acceptable salt or ester thereof is suspended in the formulation to form a suspension.

Herein, the term “comprises” and variations thereof do not have a limiting meaning where these terms appear in the description and claims. Such terms will be understood to imply the inclusion of a stated step or element, or group of steps or elements, but not the exclusion of any other step or element, or group of steps or elements. The phrase “consisting of” means including, and limited to, whatever follows the phrase “consisting of.” Thus, the phrase “consisting of” indicates that the listed elements are required or mandatory, and that no other elements may be present. The phrase “consisting essentially of” means including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may, or may not, be present depending upon whether or not they materially affect the activity or action of the listed elements. Any of the elements or combinations of elements that are recited in this specification in open-ended language (e.g., comprise and derivatives thereof), are considered to additionally be recited in closed-ended language (e.g., consist and derivatives thereof) and in partially closed-ended language (e.g., consist essentially and derivatives thereof).

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

Throughout this disclosure, singular forms such as “a,” “an,” and “the” are often used for convenience: singular forms are meant to include the plural unless the singular alone is explicitly specified or is clearly indicated by the context.

As used herein, the term “or” is generally employed in its usual sense including “and/or” unless the content clearly dictates otherwise.

The term “and/or” means one or all of the listed elements or a combination of any two or more of the listed elements.

The phrase “ambient conditions” as used herein, refers to an environment of room temperature (approximately 20° C. to 25° C.) and 30% to 60% relative humidity.

Also herein, all numbers are assumed to be modified by the term “about” and in certain embodiments, preferably, by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50). Herein, “at least” a number (e.g., at least 50) includes the number (e.g., 50). Herein, “no more than” a number (e.g., no more than 50) includes the number (e.g., 50).

Numerical ranges, for example “between x and y” or “from x to y”, include the endpoint values of x and y. Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

Some terms used in this application have special meanings, as defined herein. All other terms will be known to the skilled artisan and are to be afforded the meaning that a person of skill in the art at the time of the invention would have given them.

Elements in this specification that are referred to as “common,” “commonly used,” “conventional,” “typical,” “typically,” and the like, should be understood to be common within the context of the compositions, articles, such as inhalers and pMDIs, and methods of this disclosure: this terminology is not used to mean that these features are present, much less common, in the prior art. Unless otherwise specified, only the Background section of this Application refers to the prior art.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

The present disclosure will be described with respect to embodiments and with reference to certain drawings, but the invention is not limited thereto. The drawings described are only schematic and are non-limiting. In the drawings, the size of some of the elements can be exaggerated and not drawn to scale for illustrative purposes.

The above summary of the present disclosure is not intended to describe each disclosed embodiment or every implementation of the present disclosure. The description that follows more particularly exemplifies illustrative embodiments. In several places throughout the disclosure, guidance is provided through lists of examples, which examples may be used in various combinations. In each instance, the recited list serves only as a representative group and should not be interpreted as an exclusive or exhaustive list. Thus, the scope of the present disclosure should not be limited to the specific illustrative structures described herein, but rather extends at least to the structures described by the language of the claims, and the equivalents of those structures. Any of the elements that are positively recited in this specification as alternatives may be explicitly included in the claims or excluded from the claims, in any combination as desired. Although various theories and possible mechanisms may have been discussed herein, in no event should such discussions serve to limit the claimable subject matter.

### BRIEF DESCRIPTION OF THE DRAWINGS

The present disclosure will be described with respect to embodiments and with reference to certain drawings, but the invention is not limited thereto. The drawings described are only schematic and are non-limiting. In the drawings, the size of some of the elements can be exaggerated and not drawn to scale for illustrative purposes.

FIG. 1 is a cross-sectional side view of an inhaler including a canister containing a valve according to the present disclosure.

FIG. 2 is a detailed cross-sectional side view of the inhaler of FIG. 1.

FIG. 3 is a cross-sectional side view of a metering valve for an inhaler.

### DETAILED DESCRIPTION

The formulations of the present disclosure are suspensions (i.e., suspension formulations or suspension compositions). That is, the formulations include one or more APIs dispersed in the formulations (i.e., suspended in the propellant and often a suspension aid) to form suspensions. Herein, in a “suspension” the API is in a microparticulate solid form (typically micronized, but can also be size reduced by a multitude of other particle size reduction techniques) and dispersed in a propellant, often with other soluble or non-solubilized excipients to aid the suspension behavior of the particles. Herein, a suspension is a dispersion of particles of particulate material (e.g., API) that is visible to the unaided human eye, although there may also be a small amount of solubilized particulate material within the composition. For suspension formulations, solubilization of API is generally undesirable. In embodiments, it may be desirable to minimize solubilization of an API.

Solution and suspension formulations are fundamentally different pMDI formulation approaches. Different factors need to be considered when undertaking the development of products using either of these formulation approaches. Accordingly, it is not possible to apply the same knowledge and understanding of solution formulations to suspension formulations. Suspensions, for example, need to achieve a degree of physical stability to avoid significant separation of the physical mixture via sedimentation or creaming of the suspended particles. This can lead to poor dose to dose reproducibility. Therefore, for suspensions, the use of suspension aids to control flocculation are often used. Also, in suspensions, the resultant aerosol particle size is predominantly influenced by the geometric particle size of the microparticulate API that can change if the API particles are partially soluble in the propellant/formulation, which can lead to physical instability over time, through particle growth. Suspensions also have a potential problem with deposition of the suspended API particles on to the internal surfaces of the canister and valve, which again can cause changes to product performance over time. These problems are specific to suspensions and any teachings specific to solutions do not necessarily overcome them.

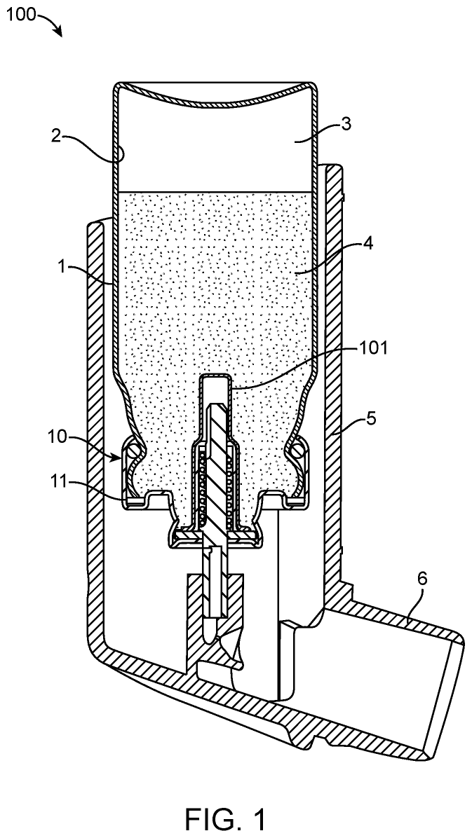

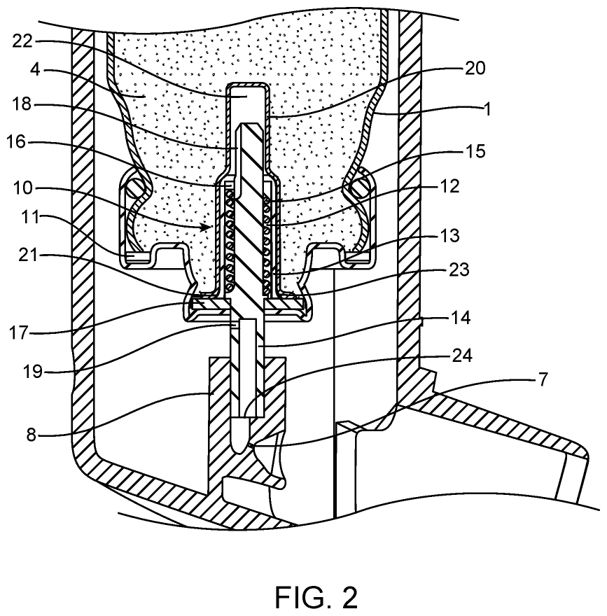

The various embodiments of formulations described herein can be utilized with any suitable inhaler. For example, FIG. 1 shows one embodiment of a metered dose inhaler 100, including an aerosol canister 1 fitted with a metered dose metering valve 10 (shown in its resting position). The metering valve 10 is typically affixed, i.e., crimped, onto the canister via a cap or ferrule 11 (typically made of aluminum or an aluminum alloy) which is generally provided as part of the valve assembly. Between the canister and the ferrule there may be one or more seals. In the embodiments shown in FIGS. 1 and 2 between the canister 1 and the ferrule 11 there are two seals including, e.g., an O-ring seal and a gasket seal.

As shown in FIG. 1, the canister/valve dispenser is typically provided with an actuator 5 including an appropriate patient port 6, such as a mouthpiece. For administration to the nasal cavities the patient port is generally provided in an appropriate form (e.g., smaller diameter tube, often sloping upwardly) for delivery through the nose. Actuators are generally made of a plastic material, for example polypropylene or polyethylene. As can be seen from FIG. 1, inner walls 2 of the canister and outer walls 101 of the portion(s) of the metering valve 10 located within the canister define a formulation chamber 3 in which aerosol formulation 4 is contained.

The valve 10 shown in FIGS. 1 and 2, includes a metering chamber 12, defined in part by an inner valve body 13, through which a valve stem 14 passes. The valve stem 14, which is biased outwardly by a compression spring 15, is in sliding sealing engagement with an inner tank seal 16 and an outer diaphragm seal 17. The valve 10 also includes a second valve body 20 in the form of a bottle emptier. The inner valve body 13 (also referred to as the “primary” valve body) defines in part the metering chamber 12. The second valve body 20 (also referred to as the “secondary” valve body) defines in part a pre-metering region or chamber besides serving as a bottle emptier.

Referring to FIG. 2, aerosol formulation 4 can pass from the formulation chamber 3 into a pre-metering chamber 22 provided between the secondary valve body 20 and the primary valve body 13 through an annular space 21 between a flange 23 of the secondary valve body 20 and the primary valve body 13. To actuate (fire) the valve 10, the valve stem 14 is pushed inwardly relative to the canister 1 from its resting position shown in FIGS. 1 and 2, allowing formulation to pass from the metering chamber 12 through a side hole 19 in the valve stem and through a stem outlet 24 to an actuator nozzle 7 then out to the patient. When the valve stem 14 is released, formulation enters into the valve 10, in particular into the pre-metering chamber 22, through the annular space 21 and thence from the pre-metering chamber through a groove 18 in the valve stem past the tank seal 16 into the metering chamber 12.

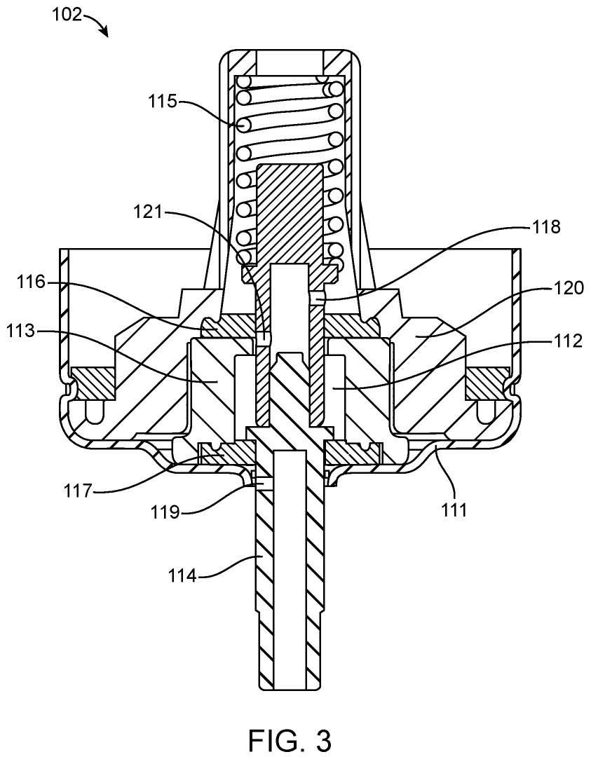

FIG. 3 shows another embodiment of a metered dose aerosol metering valve 102, different from the embodiment shown in FIGS. 1 and 2, in its rest position. The valve 102 has a metering chamber 112 defined in part by a metering tank 113 through which a stem 114 is biased outwardly by spring 115. The stem 114 is made in two parts that are push fit together before being assembled into the valve 102. The stem 114 has an inner seal 116 and an outer seal 117 disposed about it and forming sealing contact with the metering tank 113. A valve body 120 crimped into a ferrule 111 retains the aforementioned components in the valve. In use, formulation enters the metering chamber via orifices 121 and 118. The formulation's outward path from the metering chamber 112 when a dose is dispensed is via orifice 119.

The primary propellant of compositions (i.e., formulations) according to the disclosure is HFO-1234ze(E), also known as trans-1,1,1,3-tetrafluoropropene, trans-1,3,3,3-tetrafluoropropene, or trans-1,3,3,3-tetrafluoroprop-1-ene. The chemical structure of trans and cis isomers of HFO-1234ze are very different. As a result, these isomers have very different physical and thermodynamic properties. The significantly lower boiling point and higher vapor pressure of the trans (E) isomer relative to that of the cis (Z) isomer, at ambient conditions, makes the trans isomer a far more thermodynamically suitable propellant for achieving efficient pMDI atomization.

In some embodiments, the amount of HFO-1234ze(E) by weight in the composition is greater than 70%, at least 80%, greater than 80%, at least 85%, greater than 85%, at least 90%, or greater than 90%. In some embodiments, the amount of HFO-1234ze(E) by weight is between 80% and 99%, between 80% and 98%, between 80% and 95%, or between 85% and 90%. In some embodiments, HFO-1234ze(E) is essentially the sole propellant in the composition. That is, the pharmaceutical product performance parameters, such as emitted dose and emitted particle size distribution, are not significantly different than if HFO-1234ze(E) were the sole propellant in the composition. In some embodiments, the amount of HFO-1234ze(E) by weight of the total propellant in the composition is greater than 95%, greater than 98%, greater than 99%, greater than 99.5%, and greater than 99.8%.

The propellant HFO-1234ze(E) is very different from an alternative low GWP propellant HFA-152a. These two propellants have different physical, chemical, and thermodynamic properties such as boiling point, vapor pressure, water solubility, liquid density, surface tension, etc. The differences in these properties make replacing one propellant with another without significantly compromising or altering pMDI product performance difficult to achieve. For example, the thermodynamic differences in propellant boiling point and vapor pressure can significantly affect pMDI aerosolization efficiency and give rise to differences in primary and secondary atomization mechanisms. Liquid density differences between the propellants and suspended drug particles can affect suspension behavior, such as sedimentation rate. Differences in hygroscopicity between the propellants can affect moisture uptake, which could be problematic for suspension formulations, particularly if physical stability due to moisture uptake or chemical degradation in which water is involved is likely. Chemical interactions of the different propellants with drug and excipients may also be significantly different, which could affect the long-term chemical stability of the product over the intended shelf life. The two propellants interact chemically and physically with valve plastics and elastomeric components, which could give rise to differences in the types and amounts of extractables and leachables, as well as impacting mechanical valve function. The thermodynamic properties of the propellants can give rise to different droplet particle sizes due to different evaporation rates and can also result in differences in spray characteristics such as spray force, temperature, and spray duration. Historically, the transition from CFC to HFA propellants has required significant efforts to develop new approaches to reformulate and develop capable hardware to achieve appropriate pMDI product performance. That is, it was not possible to simply directly substitute one propellant for another. Changing between propellant HFA-152a to HFO-1234ze(E) in a pMDI, is equally challenging due to many of the factors highlighted above.

In some embodiments, other propellants, such as hydrofluoroalkanes, including HFA-134a, HFA-227 (1,1,1,2,3,3,3-heptafluoropropane), or HFA-152a, may be included as a minor component. Still other propellants that may be included as a minor component include other hydrofluroroolefins, including HFO-1234yf and HFO-1234ze(Z) (i.e., cis-HFO-1234ze). Amounts of such secondary propellants can include 0.1% to 20%, 0.1% to 10%, 0.1% to 5%, 0.1% to 0.5%, 5% to 20%, or 10% to 20%, by weight, of the composition (i.e., formulation). Thus, in some embodiments, the differences between HFA-152a and HFO-1234ze(E) discussed herein can be utilized to advantage by using a minor amount of HFA-152a. For example, in some embodiments, a minor amount of HFA-152a may be used to inhibit deposition of API particles on the surfaces of the metered dose inhaler that are contacted by the formulation as it passes from the canister in which it is stored to the nozzle outlet.

The total amount of composition is desirably selected so that at least a portion of the propellant in the canister is present as a liquid after a predetermined number of medicinal doses have been delivered. The predetermined number of doses may be 5 to 200, 30 to 200, 60 to 200, 60 to 120, 60, 120, 200, or any other number of doses. The total amount of composition in the canister may be from 1.0 grams (g) to 30.0 g. 2.0 g to 20.0 g. or 5.0 to 10.0 g. The total amount of composition is typically selected to be greater than the product of the predetermined number of doses and the metering volume of the metering valve. In some embodiments, the total amount of composition is greater than 1.1 times, greater than 1.2 times, greater than 1.3 times, greater than 1.4 times, or greater than 1.5 times the product of the predetermined number of doses and the metering volume of the metering valve. This typically ensures that the amount of each dose remains relatively constant through the life of the inhaler.

The active pharmaceutical ingredient (API) may be a drug, vaccine, DNA fragment, hormone, other treatment, or a combination of any two or more APIs. In certain embodiments, the formulations may include at least two (in certain embodiments, two or three, and in certain embodiments, two) APIs in suspension.

For preparation of suspension formulations, the API is preferably provided as a micronized powder. However, it should be apparent to one of ordinary skill in the art that other forms of API may be suitable for preparation of suspension formulations consistent with this disclosure.

Exemplary APIs can include those for the treatment of respiratory disorders, e.g., a bronchodilator, such as a short or long acting beta agonist, an anti-inflammatory (e.g., a corticosteroid), an anti-allergic, an anti-asthmatic, an antihistamine, a TYK inhibitor, or an anticholinergic agent. Exemplary APIs can include salbutamol (i.e., albuterol), levalbuterol, terbutaline, ipratropium, oxitropium, tiotropium, beclomethasone, flunisolide, budesonide, mometasone, ciclesonide, cromolyn sodium, nedocromil sodium, ketotifen, azelastine, ergotamine, cyclosporine, aclidinium, umeclidinium, glycopyrronium (i.e., glycopyrrolate), salmeterol, fluticasone, formoterol, procaterol, indacaterol, carmoterol, milveterol, olodaterol, vilanterol, abediterol, omalizumab, zileuton, insulin, pentamidine, calcitonin, leuprolide, alpha-I-antitrypsin, interferon, triamcinolone, nintedanib, a pharmaceutically acceptable salt or ester of any of the listed drugs, or a mixture of any of the listed drugs, their pharmaceutically acceptable salts or their pharmaceutically acceptable esters. For fluticasone, exemplary esters include propionate or furoate: for beclomethasone, an exemplary ester is propionate: and for mometasone, an exemplary ester is furoate.

In all embodiments, the API(s) are dispersed or suspended in the formulation (i.e., as a suspension). In the event a combination of two or more APIs are used, all of the APIs are suspended. Where API is present in particulate form, i.e., suspended, it will generally have a mass median aerodynamic diameter in the range of 1 micrometer (μm) to 10 μm, preferably 1 μm to 5 μm.

In one embodiment, the formulation has salbutamol (i.e., albuterol) or a pharmaceutically acceptable salt or ester thereof as the sole API, more particularly salbutamol sulfate (i.e., albuterol sulfate).

In one embodiment, the formulation has budesonide or a pharmaceutically acceptable salt or ester thereof as the sole API.

In one embodiment, the formulation has mometasone or a pharmaceutically acceptable salt or ester thereof as the sole API, more particularly mometasone furoate

In one embodiment, the formulation has fluticasone or a pharmaceutically acceptable salt or ester thereof as the sole API, more particularly fluticasone propionate.

The amount of API may be determined by the required dose per actuation and the pMDI metering valve size, that is, the size of the metering chamber, which may be between 5 microliters (μL or mcl) and 200 microliters, between 25 microliters and 200 microliters, between 25 microliters and 150 microliters, between 25 microliters and 100 microliters, or between 25 microliters and 65 microliters. The concentration of each API is typically from 0.0008% to 3.4% by weight, or 0.01% to 1.0% by weight, sometimes from 0.05% to 0.5% by weight, and as such, the medicament makes up a relatively small percentage of the total composition.

In certain embodiments, typical formulations of the present disclosure include the API in an amount of at least 0.001 milligram per actuation (mg/actuation) (1 microgram (μg, mcg) per actuation), or at least 0.01 mg/actuation (10 μg/actuation). In certain embodiments, typical formulations of the present disclosure include the API in an amount of less than 0.5 mg/actuation (500 μg/actuation).

In embodiments, typical formulations of the present disclosure include the API in an amount of at least 1 μg/actuation, at least 10 μg/actuation, at least 50 μg/actuation, at least 100 μg/actuation, at least 150 μg/actuation, at least 200 μg/actuation, at least 300 μg/actuation, or at least 400 μg/actuation. In embodiments, typical formulations of the present disclosure include the API in an amount of less than 500 μg/actuation, at most 400 μg/actuation, at most 300 μg/actuation or at most 200 μg/actuation. In some preferred embodiments, formulations of the present disclosure include the API in an amount of 80 μg/actuation to 120 μg/actuation.

In some embodiments, additional components (e.g., excipients) beyond propellant and API can be added to the formulation. These components may have various uses and functions, including, but not limited to, facilitating formation of a suspension, stabilizing a suspension, and/or aiding in chemical stabilization of API or other components.

In some embodiments a cosolvent is included. One particularly useful cosolvent is ethanol. In one aspect, ethanol may aid in directly or indirectly stabilizing the suspension whereas the suspension may not be stable in the absence of the ethanol. In certain embodiments, when used in suspension formulations, ethanol may be in amounts on a weight percent basis of the total formulation of at least 0.1%, at least 0.2% at least 0.4%, at least 0.5%, at least 1%, at least 2%, or at least 3%. In certain embodiments, when used in suspension formulations, ethanol may be in amounts on a weight percent basis of the total formulation of up to 20%, 15%, up to 12%, up to 10%, up to 8%, up to 5%, or up to 2%.

In certain embodiments, when used in suspension formulations, ethanol may be in amounts on a weight percent basis of the total formulation of between 0.1% and 20%, between 0.1% and 15%, between 0.2% and 15%, between 0.2% and 10%, between 0.2% and 5%, between 0.4% and 10%, between 0.4% and 5%, between 0.5% and 20%, between 0.5% and 15%, between 0.5% and 10%, between 0.5% and 5%, between 0.5% and 2%, between 1% and 12%, between 1% and 10%, between 2% and 10%, between 2% and 5%, or between 3% and 8%.

The use of ethanol in embodiments of HFO-1234ze(E) suspension formulations of the present disclosure may advantageously reduce deposition of suspended API particles on to internal canister and/or valve surfaces, which can in turn minimize the impact of such deposition on reduction of delivered dose relative to the anticipated delivered dose and/or improve overall through unit life dosing consistency.

However, in some embodiments, increasing concentrations of ethanol could disadvantageously increase the solubility of the suspended API particles and facilitate undesirable particle growth. The amount of ethanol to be included in a given HFO-1234ze(E) suspension formulation may be titrated for an API of interest to promote overall delivered dose efficiency and/or through unit life dosing consistency, while minimizing deposition.

In some embodiments of the present disclosure, inclusion of ethanol in HFO-1234ze(E) suspensions beyond an effective minimal level to effectively reduce API deposition and achieve effective overall dose delivery, will adversely impact on other important suspension formulation performance characteristics, such as increasing aerodynamic particle size, and reducing fine particle fraction (FPF) and fine particle mass (FPM).

In some embodiments a surfactant can also be used to facilitate suspension of particles in the formulation. However, surfactant-free formulations can be advantageous for some purposes, and surfactant is not required unless otherwise specified.

Any pharmaceutically acceptable surfactant can be used. Exemplary surfactants include oleic acid, sorbitan monooleate, sorbitan trioleate, soya lecithin, polyethylene glycol, polyvinylpyrrolidone, or combinations thereof. When polyvinylpyrrolidone is employed, it can have any suitable molecular weight. Examples of suitable weight average molecular weights are from 10 kilodaltons to 100 kilodaltons, 10 kilodaltons to 50 kilodaltons, 10 kilodaltons to 40 kilodaltons, 10 kilodaltons to 30 kilodaltons, or 10 kilodaltons to 20 kilodaltons. When polyethylene glycol is employed, it can be any suitable molecular weight. Examples of suitable weight average molecular weights are from 300 daltons to 1000 daltons. In some embodiments, PEG 1000 and PEG 300 are employed. When used, the amount of surfactant on a weight percent basis of the total formulation is between 0.0001% and 1%, between 0.001% and 0.1%, or between 0.01% and 0.1%.

In certain embodiments, small amounts of water may be in suspension formulations. Preferably, however, added water is not used in making suspension formulations of the present disclosure.

In certain embodiments, compositions of the present disclosure preferably display chemical stability such that acceptable levels of degradation products are present in the finished product for at least 24 months, and often from 24 to 36 months under required storage conditions.

Returning to FIG. 1, in use, the patient actuates the inhaler 100 by pressing downwardly on the canister 1. This moves the canister 1 into the body of the actuator 5 and presses the valve stem 14 against the actuator stem socket 8 resulting in the canister metering valve 10 opening and releasing a metered dose of composition that passes through the actuator nozzle 7 and exits the mouthpiece 6 into the patient's mouth. It should be understood that other modes of actuation, such as breath-actuation, may be used as well and would operate as described with the exception that the force to depress the canister would be provided by the device, for instance by a spring or a motor-driven screw, in response to a triggering event, such as patient inhalation.

Devices that may be used with medicament compositions of the present disclosure include those described in U.S. Pat. No. 6,032,836 (Hiscocks et al.), U.S. Pat. No. 9,010,329 (Hansen), and U.K. Patent GB 2544128 B (Friel).

The metered dose inhaler can include a dose counter for counting the number of doses. Suitable dose counters are known in the art, and are described in, for example, U.S. Pat. No. 8,740,014 (Purkins et al.): U.S. Pat. No. 8,479,732 (Stuart et al.): and U.S. Pat. No. 8,814,035 (Stuart), and U.S. Patent Application Publication No. 2012/0234317 (Stuart), all of which are incorporated by reference in their entirety with respect to their disclosures of dose counters.

One exemplary dose counter, which is described in detail in U.S. Pat. No. 8,740,014 (Purkins et al., hereby incorporated by reference in its entirety for its disclosure of the dose counter) has a fixed ratchet element and a trigger element that is constructed and arranged to undergo reciprocal movement coordinated with the reciprocal movement between an actuation element in an inhaler and the dose counter. The reciprocal movement can include an outward stroke (outward being with respect to the inhaler) and a return stroke. The return stroke returns the trigger element to the position that it was in prior to the outward stroke. A counter element is also included in this type of dose counter. The counter element is constructed and arranged to undergo a predetermined counting movement each time a dose is dispensed. The counter element is biased towards the fixed ratchet and trigger elements and is capable of counting motion in a direction that is substantially orthogonal to the direction of the reciprocal movement of the trigger element.

The counter element in the above-described dose counter includes a first region for interacting with the trigger member. The first region includes at least one inclined surface that is engaged by the trigger member during the outward stroke of the trigger member. This engagement during the outward stroke causes the counter element to undergo a counting motion. The counter element also includes a second region for interacting with the ratchet member. The second region includes at least one inclined surface that is engaged by the ratchet element during the return stroke of the trigger element causing the counter element to undergo a further counting motion, thereby completing a counting movement. The counter element is normally in the form of a counter ring, and is advanced partially on the outward stroke of the trigger element, and partially on the return stroke of the trigger element. As the outward stroke of the trigger can correspond to the depression of a valve stem that causes firing of the valve (and, in the case of a metered dose inhaler, also meters the contents) and the return stroke can correspond to the return of the valve stem to its resting position, this dose counter allows for precise counting of doses.

Another suitable dose counter, which is described in detail in U.S. Pat. No. 8,479,732 (Stuart et al., hereby incorporated by reference in its entirety for its disclosure of dose counters) is specially adapted for use with a metered dose inhaler. This dose counter includes a first count indicator having a first indicia bearing surface. The first count indicator is rotatable about a first axis. The dose counter also includes a second count indicator having a second indicia bearing surface. The second count indicator is rotatable about a second axis. The first and second axes are disposed such that they form an obtuse angle. The obtuse angle mentioned above can be any obtuse angle, but is advantageously 125 to 145 degrees. The obtuse angle permits the first and second indicia bearing surface to align at a common viewing area to collectively present at least a portion of a medication dosage count. One or both of the first and second indicia bearing surfaces can be marked with digits, such that when viewed together through the viewing area the numbers provide a dose count. For example, one of the first and second indicia bearing surface may have “hundreds” and “tens” place digits, and the other with “ones” place digits, such that when read together the two indicia bearing surfaces provide a number between 000 and 999 that represents the dose count.

Yet another suitable dose counter is described in U.S. Patent Application Publication No. 2012/0234317 (Stuart, hereby incorporated by reference in its entirety for its disclosure of dose counters). Such a dose counter includes a counter element that undergoes a predetermined counting motion each time a dose is dispensed. The counting motion can be vertical or essentially vertical. A count indicating element is also included. The count indicating element, which undergoes a predetermined count indicating motion each time a dose is dispensed, includes a first region that interacts with the counter element.

The counter element has regions for interacting with the count indicating element. Specifically, the counter element includes a first region that interacts with a count indicating element. The first region includes at least one surface that it engaged with at least one surface of the first region of the aforementioned count indicating element. The first region of the counter element and the first surface of the count inducing element are disposed such that the count indicating member completes a count indicating motion in coordination with the counting motion of the counter element, during and induced by the movement of the counter element, the count inducing element undergoes a rotational or essentially rotational movement. In practice, the first region of the counter element or the counter indicating element can include, for example, one or more channels. A first region of the other element can include one or more protrusions adapted to engage with said one or more channels.

Yet another dose counter is described in U.S. Pat. No. 8,814,035 (Stuart, hereby incorporated by reference in its entirety for its disclosure of dose counters). Such a dose counter is specially adapted for use with an inhaler with a reciprocal actuator operating along a first axis. The dose counter includes an indicator element that is rotatable about a second axis. The indicator element is adapted to undergo one or more predetermined count-indicating motions when one or more doses are dispensed. The second axis is at an obtuse angle with respect to the first axis. The dose counter also contains a worm rotatable about a worm axis. The worm is adapted to drive the indicator element. It may do this, for example, by containing a region that interacts with and enmeshes with a region of the indicator element. The worm axis and the second axis do not intersect and are not aligned in a perpendicular manner. The worm axis is also, in most cases, not disposed in coaxial alignment with the first axis. However, the first and second axes may intersect.

At least one of the various internal components of an inhaler, such as a metered dose inhaler, as described herein, such as one or more of the canister, valve, gaskets, seals, or O-rings, can be coated with one or more coatings. Some of these coatings provide a low surface energy. Such coatings are not always required because they are not always necessary for the successful operation of all inhalers. Thus, some metered dose inhalers do not include coated internal components

Some coatings that can be used are described in U.S. Pat. No. 8,414,956 (Jinks et al.), U.S. Pat. No. 8,815,325 (David et al.), and United States Patent Application Publication No. 2012/0097159 (Iyer et al.), all of which are incorporated by reference in their entireties for their disclosure of coatings for inhalers and inhaler components. Other coatings, such as fluorinated ethylene propylene resins, or FEP, are also suitable. FEP is particularly suitable for use in coating canisters.

A first acceptable coating can be provided by the following method:
a) providing one or more component of the inhaler, such as the metered dose inhaler,
b) providing a primer composition including a silane having two or more reactive silane groups separated by an organic linker group,
c) providing a coating composition including an at least partially fluorinated compound,
d) applying the primer composition to at least a portion of the surface of the component,
e) applying the coating composition to the portion of the surface of the component after application of the primer composition.

The at least partially fluorinated compound will usually include one or more reactive functional groups, with the at least one reactive functional group usually being a reactive silane group, for example a hydrolysable silane group or a hydroxysilane group.

Such reactive silane groups allow reaction of the partially fluorinated compound with one or more of the reactive silane groups of the primer. Often such reaction will be a condensation reaction.

One exemplary silane that can be used has the formula

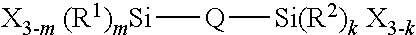

wherein R¹ and R² are independently selected univalent groups, X is a hydrolysable or hydroxy group, m and k are independently 0, 1, or 2 and Q is a divalent organic linking group.

Useful examples of such silanes include one or a mixture of two or more of 1,2-bis(trialkoxysilyl) ethane, 1,6-bis(trialkoxysilyl) hexane, 1,8-bis(trialkoxysilyl) octane, 1,4-bis(trialkoxysilylethyl)benzene, bis(trialkoxysilyl)itaconate, and 4,4′-bis(trialkoxysilyl)-1,1′-diphenyl, wherein any trialkoxy group may be independently trimethoxy or triethoxy.

The coating solvent usually includes an alcohol or a hydrofluoroether.

If the coating solvent is an alcohol, preferred alcohols are C₁ to C₄ alcohols, in particular, an alcohol selected from ethanol, n-propanol, or isopropanol or a mixture of two or more of these alcohols.

If the coating solvent is an hydrofluoroether, it is preferred if the coating solvent includes a C₄ to C₁₀ hydrofluoroether. Generally, the hydrofluoroether will be of formula

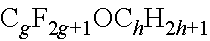

wherein g is 2, 3, 4, 5, or 6 and h is 1, 2, 3, or 4. Examples of suitable hydrofluoroethers include those selected from the group consisting of methyl heptafluoropropylether, ethyl heptafluoropropylether, methyl nonafluorobutylether, ethyl nonafluorobutylether and mixtures thereof.

The polyfluoropolyether silane can be of the formula

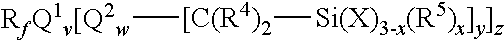

wherein:
   R^{f} is a polyfluoropolyether moiety;
   Q¹ is a trivalent linking group;
   each Q² is an independently selected organic divalent or trivalent linking group;
   each R⁴ is independently hydrogen or a C₁₋₄ alkyl group;
   each X is independently a hydrolysable or hydroxyl group;
   R⁵ is a C₁₋₈ alkyl or phenyl group;
   v and w are independently 0 or 1, x is 0 or 1 or 2: y is 1 or 2; and z is 2, 3, or 4.

The polyfluoropolyether moiety R^{f} can include perfluorinated repeating units selected from the group consisting of —(CₙF₂ₙO)—, —(CF(Z)O)—, —(CF(Z)CₙF₂ₙO)—, —(CₙF₂ₙCF(Z)O)—, —(CF₂CF(Z)O)—, and combinations thereof; wherein n is an integer from 1 to 6 and Z is a perfluoroalkyl group, an oxygen-containing perfluoroalkyl group, a perfluoroalkoxy group, or an oxygen-substituted perfluoroalkoxy group, each of which can be linear, branched, or cyclic, and have 1 to 5 carbon atoms and up to 4 oxygen atoms when oxygen-containing or oxygen-substituted, and wherein for repeating units including Z the number of carbon atoms in sequence is at most 6. In particular, n can be an integer from 1 to 4, more particularly from 1 to 3. For repeating units including Z the number of carbon atoms in sequence may be at most four, more particularly at most 3. Usually, n is 1 or 2 and Z is a —CF, group, more wherein z is 2, and R^{f} is selected from the group consisting of —CF₂O(CF₂O)ₘ(C₂F₄O)ₚCF₂—, —CF(CF₃)O(CF(CF₃)CF₂O)ₚCF(CF₃)—, —CF₂O(C₂F₄O)ₚCF₂—, —(CF₂)₃O(C₄F₈O)ₚ(CF₂)₃—; —CF(CF₃)—(OCF₂CF(CF₃))ₚO—CₜF₂ₜ—O(CF(CF₃)CF₂O)ₚCF(CF₃)—, wherein t is 2, 3 or 4 and wherein m is 1 to 50, and p is 3 to 40.

A cross-linking agent can be included. Exemplary cross-linking agents include tetramethoxysilane: tetraethoxysilane: tetrapropoxysilane: tetrabutoxysilane: methyl triethoxysilane: dimethyldiethoxysilane: octadecy ltriethoxysilane: 3-glycidoxy-propyltrimethoxysilane: 3-glycidoxy-propyltriethoxysilane: 3-aminopropyl-trimethoxysilane: 3-aminopropyl-triethoxysilane: bis(3-trimethoxysilylpropyl) amine: 3-aminopropyl tri(methoxyethoxyethoxy) silane: N-(2-aminoethyl)3-aminopropyltrimethoxysilane: bis(3-trimethoxysilylpropyl) ethylenediamine: 3-mercaptopropyltrimethoxysilane: 3-mercaptopropyltriethoxysilane: 3-trimethoxysilyl-propylmethacry late: 3-triethoxysilypropylmethacry late: bis(trimethoxysilyl) itaconate: allyltriethoxysilane: allyltrimethoxysilane: 3-(N-allylamino)propyltrimethoxysilane: vinyltrimethoxysilane: vinyltriethoxysilane; and mixtures thereof.

The component to be coated can be pre-treated before coating, such as by cleaning. Cleaning can be by way of a solvent, such as a hydrofluoroether, e.g., HFE-72DE, or an azeotropic mixture of 70% w/w (i.e., weight percent) trans-dichloroethylene: 30% w/w of a mixture of methyl and ethyl nonafluorobutyl and nonafluoroisobutyl ethers.

The above-described first acceptable coating is particularly useful for coating valves components, including one or more of valve stems, bottle emptiers, springs, and tanks. This coating system can be used with any type of inhaler and any formulation described herein.

In some embodiments the actuator nozzle is sized so as to optimize the fine particle fraction (FPF) and/or respirable dose delivered of the formulation within the canister. In some embodiments the cross-sectional shape of the actuator nozzle is essentially circular or circular and has a predetermined diameter. In some embodiments where the cross-sectional shape of the actuator nozzle is non-circular, for example oval, an effective diameter may be determined by taking an average over the distances spanning the opening (e.g., the average of major and minor axes of an ellipse).

In some embodiments the exit orifice (effective diameter) of the actuator nozzle may be 0.08 mm or greater, 0.10 mm or greater, 0.12 mm or greater, 0.15 mm or greater, 0.175 mm or greater, 0.225 mm or greater 0.3 mm or greater, or 0.4 mm or greater. In some embodiments the exit orifice (effective diameter) of the actuator nozzle may be 0.5 mm or less, 0.4 mm or less, 0.3 mm or less, 0.225 mm or less, 0.175 mm or less, or 0.15 mm or less. In some embodiments the exit orifice (effective diameter) of the actuator nozzle may be 0.12 mm to 0.5 mm, 0.12 mm to 0.4 mm, 0.12 mm to 0.3 mm, 0.12 mm to 0.225 mm, 0.12 mm to 0.175 mm, or 0.12 mm to 0.15 mm. In some embodiments the exit orifice (effective diameter) of the actuator nozzle may be 0.15 mm to 0.5 mm, 0.15 mm to 0.4 mm, 0.15 mm to 0.3 mm, 0.15 mm to 0.225 mm, or 0.15 mm to 0.175 mm. In some embodiments the exit orifice (effective diameter) of the actuator nozzle may be 0.175 mm to 0.5 mm, 0.175 mm to 0.4 mm, 0.175 mm to 0.3 mm, or 0.175 mm to 0.225 mm. In some embodiments the exit orifice (effective diameter) of the actuator nozzle may be 0.225 mm to 0.5 mm, 0.225 mm to 0.4 mm, or 0.225 mm to 0.3 mm. In some embodiments the exit orifice (effective diameter) of the actuator nozzle may be 0.3 mm to 0.5 mm or 0.3 mm to 0.4 mm. In some embodiments the exit orifice (effective diameter) of the actuator nozzle may be 0.4 mm to 0.5 mm. It may be particularly advantageous to use smaller actuator nozzle sizes as described above (e.g., diameter between 0.12 mm (120 μm) and 0.225 mm (225 μm), or between 0.175 mm (175 μm) and 0.225 mm (225 μm)) in conjunction with formulations where the API is present as a suspension. This may aid in increasing the fine particle fraction of the emitted dose.

It should be appreciated by one of ordinary skill in the art that a given actuator nozzle exit orifice may not be suitable for delivery of any formulation, and that selection of a suitable actuator nozzle exit orifice for a given formulation involves considerable effort.

In some embodiments, the MDI is manufactured by pressure filling. In pressure filling, the powdered medicament, optionally combined with one or more excipients (e.g., cosolvents), is placed in a suitable aerosol container (i.e., canister) capable of withstanding the vapor pressure of the propellant and fitted with a metering valve prior to filling. The propellant is then forced as a liquid through the valve into the container. In an alternate process of pressure filling, the particulate drug is combined in a process vessel with propellant and optionally one or more excipients (e.g., cosolvents), and the resulting drug suspension is transferred through the metering valve fitted to a suitable MDI container.

In some embodiments, the MDI is manufactured by cold filling. In cold filling, the powdered medicament, propellant which is chilled below its boiling point and, optionally, one or more excipients (e.g., co-solvents) are added to the MDI container. In addition, a metering valve is fitted to the container post filling.

For both pressure filling and cold filling processes, additional steps, such as mixing, sonication, and homogenization may be optionally employed.

### Embodiments

Embodiment 1 is a metered dose inhaler comprising: a metering valve: a canister: and an actuator comprising an actuator nozzle: wherein the canister comprises a formulation, the formulation comprising greater than 70% by weight of propellant HFO-1234ze(E), ethanol, and at least one active pharmaceutical ingredient suspended in the formulation to form a suspension.

Embodiment 2 is the inhaler of embodiment 1, wherein the active pharmaceutical ingredient is selected from beta agonists (short- or long-acting beta agonists), corticosteroids, anticholinergic agents, TYK inhibitors, and combinations thereof.

Embodiment 3 is the inhaler of embodiment 1, wherein the active pharmaceutical ingredient comprises a corticosteroid.

Embodiment 4 is the inhaler of embodiment 2 or 3, wherein the corticosteroid is selected from beclomethasone, budesonide, mometasone, ciclesonide, flunisolide, and fluticasone.

Embodiment 5 is the inhaler of embodiment 1, wherein the active pharmaceutical ingredient comprises an anticholinergic agent.

Embodiment 6 is the inhaler of embodiment 2 or 5, wherein the anticholinergic agent is selected from ipratropium, tiotropium, aclidinium, umeclidinium, and glycopyrronium.

Embodiment 7 is the inhaler of embodiment 1, wherein the active pharmaceutical ingredient comprises a beta agonist (short- or long-acting).

Embodiment 8 is the inhaler of embodiment 2 or 7, wherein the beta agonist (short or long-acting beta agonist) is selected from salbutamol, levalbuterol, salmeterol, formoterol, indacaterol, olodaterol, vilanterol, and abediterol.

Embodiment 9 is the inhaler of any preceding embodiment, wherein the formulation comprises at least two (in some embodiments, two or three, and in some embodiments, two) active pharmaceutical ingredients.

Embodiment 10 is the inhaler of embodiment 9, wherein one active pharmaceutical ingredient is a short- or long-acting beta agonist and one active pharmaceutical ingredient is a corticosteroid.

Embodiment 11 is the inhaler of embodiment 10, wherein the formulation further comprises an anticholinergic agent.

Embodiment 12 is a metered dose inhaler comprising: a metering valve: a canister: and an actuator comprising an actuator nozzle: wherein the canister comprises a formulation, the formulation comprising a propellant comprising HFO-1234ze(E), ethanol, and an active pharmaceutical ingredient comprising fluticasone or a pharmaceutically acceptable salt or ester thereof, wherein the fluticasone or pharmaceutically acceptable salt or ester thereof is suspended in the formulation to form a suspension.

Embodiment 13 is the inhaler of embodiment 12, wherein the fluticasone or a pharmaceutically acceptable salt or ester thereof is the sole active pharmaceutical ingredient.

Embodiment 14 is the inhaler of embodiment 12 or 13, wherein the fluticasone or a pharmaceutically acceptable salt or ester thereof is fluticasone propionate.

Embodiment 15 is the inhaler of any of embodiments 12 or 14, wherein the formulation further comprises formoterol or a pharmaceutically acceptable salt or ester thereof.

Embodiment 16 is the inhaler of embodiment 15, wherein the formoterol or a pharmaceutically acceptable salt or ester thereof is formoterol fumarate.

Embodiment 17 is a metered dose inhaler comprising: a metering valve: a canister: and an actuator comprising an actuator nozzle: wherein the canister comprises a formulation, the formulation comprising a propellant comprising HFO-1234ze(E), ethanol, and an active pharmaceutical ingredient comprising salbutamol or a pharmaceutically acceptable salt or ester thereof, wherein the salbutamol or pharmaceutically acceptable salt or ester thereof is suspended in the formulation to form a suspension.

Embodiment 18 is the inhaler of embodiment 17, wherein the salbutamol or a pharmaceutically acceptable salt or ester thereof is the sole active pharmaceutical ingredient.

Embodiment 19 is the inhaler of embodiment 17 or 18, wherein the salbutamol or a pharmaceutically acceptable salt or ester thereof is salbutamol sulfate.

Embodiment 20 is a metered dose inhaler comprising: a metering valve: a canister: and an actuator comprising an actuator nozzle: wherein the canister comprises a formulation, the formulation comprising a propellant comprising HFO-1234ze(E), ethanol, and an active pharmaceutical ingredient comprising mometasone or a pharmaceutically acceptable salt or ester thereof, wherein the mometasone or pharmaceutically acceptable salt or ester thereof is suspended in the formulation to form a suspension.

Embodiment 21 is the inhaler of embodiment 20, wherein the mometasone or a pharmaceutically acceptable salt or ester thereof is the sole active pharmaceutical ingredient.

Embodiment 22 is the inhaler of embodiment 20 or 21, wherein the mometasone or a pharmaceutically acceptable salt or ester thereof is mometasone furoate.

Embodiment 23 is a metered dose inhaler comprising: a metering valve: a canister: and an actuator comprising an actuator nozzle: wherein the canister comprises a formulation, the formulation comprising a propellant comprising HFO-1234ze(E), ethanol, and an active pharmaceutical ingredient comprising budesonide or a pharmaceutically acceptable salt or ester thereof, wherein the budesonide or pharmaceutically acceptable salt or ester thereof is suspended in the formulation to form a suspension.

Embodiment 24 is the inhaler of embodiment 23, wherein the budesonide or a pharmaceutically acceptable salt or ester thereof is the sole active pharmaceutical ingredient.

Embodiment 25 is the inhaler of any of the preceding embodiments, wherein the amount of ethanol by weight of the total formulation is between 0.2% and 15%.

Embodiment 26 is the inhaler of embodiment 25, wherein the amount of ethanol by weight of the total formulation is between 0.2% and 10%.

Embodiment 27 is the inhaler of embodiment 26, wherein the amount of ethanol by weight of the total formulation is between 0.2% and 5%.

Embodiment 28 is the inhaler of embodiment 27, wherein the amount of ethanol by weight of the total formulation is between 0.4% and 5%.

Embodiment 29 is the inhaler of embodiment 27, wherein the amount of ethanol by weight of the total formulation is between 2% and 5%.

Embodiment 30 is the inhaler of any preceding embodiment, wherein the formulation comprises the active pharmaceutical ingredient in an amount of greater than 1 μg/actuation.

Embodiment 31 is the inhaler of embodiment 30, wherein the formulation comprises the active pharmaceutical ingredient in an amount of up to 0.5 mg/actuation.

Embodiment 32 is the inhaler of any preceding embodiment, wherein HFO-1234ze(E) is the sole propellant.

Embodiment 33 is the inhaler of any of embodiments 1 to 31, wherein the propellant comprises HFO-1234ze(E) and another hydrofluroroolefin or a hydrofluoroalkane.

Embodiment 34 is the inhaler of embodiment 33, wherein the formulation includes the other hydrofluroroolefin or hydrofluoroalkane in an amount of 0.1% to 20% (0.1 to 10%, 0.1% to 5%, 0.1% to 0.5%, 5% to 20%, or 10% to 20%) by weight, of the formulation.

Embodiment 35 is the inhaler of embodiment 33 or 34, wherein the formulation includes a hydrofluoroalkane in an amount of 5% to 20% by weight, of the formulation.

Embodiment 36 is the inhaler of embodiment 35, wherein the formulation includes a hydrofluoroalkane in an amount of 10% to 20% by weight, of the formulation.

Embodiment 37 is the inhaler of any of embodiments 33 to 36, wherein the propellant comprises HFO-1234ze(E) and HFA-152a.

Embodiment 38 is the inhaler of any preceding embodiment, wherein the formulation further comprises a surfactant.

Embodiment 39 is the inhaler of embodiment 38, wherein the surfactant is selected from oleic acid, sorbitan monooleate, sorbitan trioleate, soya lecithin, polyethylene glycol, polyvinylpyrrolidone, and a combination thereof.

Embodiment 40 is the inhaler of embodiment 38 or 39, wherein the formulation comprises a surfactant on a weight percent basis of the total formulation of between 0.0001% and 1%.

Embodiment 41 is the inhaler of embodiment 40, wherein the formulation comprises a surfactant on a weight percent basis of the total formulation of between 0.001% and 0.1%.

Embodiment 42 is the inhaler of embodiment 41, wherein the formulation comprises a surfactant on a weight percent basis of the total formulation of between 0.01% and 0.1%.

Embodiment 43 is the inhaler of any preceding embodiment, wherein the metering valve comprises a metering chamber having a size between 25 microliters and 200 microliters.

Embodiment 44 is the inhaler of 43, wherein the metering chamber of the metering valve has a size between 25 microliters and 100 microliters.

Embodiment 45 is the inhaler of any of embodiments 12 to 44, wherein the formulation comprises greater than 70% by weight of propellant HFO-1234ze(E).

Embodiment 46 is the inhaler of any of embodiments 1 to 45, wherein the formulation comprises greater than 80% by weight of propellant HFO-1234ze(E).

Embodiment 47 is the inhaler of embodiment 46, wherein the formulation comprises greater than 85% by weight of propellant HFO-1234ze(E).

Embodiment 48 is the inhaler of embodiment 47, wherein the formulation comprises greater than 90% by weight of propellant HFO-1234ze(E).

Embodiment 49 is the inhaler of any preceding embodiment, wherein the amount of HFO-1234ze(E) by weight of the total propellant in the formulation is greater than 95%.

Embodiment 50 is the inhaler of embodiment 49, wherein the amount of HFO-1234ze(E) by weight of the total propellant in the formulation is greater than 99%.

Embodiment 51 is the inhaler of any preceding embodiment, wherein the actuator exit orifice diameter is 0.12 mm to 0.5 mm.

Embodiment 52 is the inhaler of embodiment 51, wherein the actuator exit orifice diameter is 0.15 mm to 0.4 mm.

Embodiment 53 is the inhaler of embodiment 52, wherein the actuator exit orifice diameter is 0.175 mm to 0.4 mm.

Embodiment 54 is the inhaler of any preceding embodiment, wherein the amount of formulation in the canister is 1 mL to 30 mL.

Embodiment 55 is the inhaler of any preceding embodiment, wherein the canister contains a predetermined number of doses that is from 30 to 200.

### EXAMPLES

### Comparative Example 1: Delivered Dose of Salbutamol Sulfate Suspensions in HFA-134a, HFA-152a, or HFO-1234Ze(E) with and without Added Ethanol

In this experiment, suspensions of micronized salbutamol sulfate in either HFA-152a (1,1-difluoroethane) or HFO-1234ze(E) (1,3,3,3-tetrafluoropropene) were prepared. Each suspension included an amount of salbutamol sulfate (1.91 mg/mL) to provide a nominal dose of 100 μg/actuation. Suspensions were prepared with each propellant and either 0% or 5% ethanol by weight for a total of four suspensions. Each suspension was filled into FEP-coated canisters and tested with a KINDEVA actuator with an exit diameter orifice of 0.4 mm. A commercially available suspension of salbutamol sulfate in HFA-134a (1,1,1,2-tetrafluoroethane) with 0% ethanol was tested as a comparison. Uniformity of Delivered Dose (UoDD) was measured for each suspension. Data from this testing is shown in Table 1 below.

TABLE 1

| | Uniformity of delivered dose (μg/actuation) | | | |
|---|---|---|---|---|
| Suspension | Start | Middle | End | Overall |
| Composition | of life | of life | of life | mean |
| HFA-134a, | 83.3 | 78.2 | 67.0 | 76.4 |
| 0% ethanol | | | | |
| HFA-152a, | 82.3 | 81.7 | 76.6 | 80.3 |
| 0% ethanol | | | | |
| HFA-152a, | 92.9 | 96.0 | 89.6 | 93.1 |
| 5% ethanol | | | | |
| HFO-1234ze(E), | 28.5 | 53.9 | 56.9 | 46.4 |
| 0% ethanol | | | | |
| HFO-1234ze(E), | 83.3 | 84.4 | 81.7 | 83.3 |
| 5% ethanol | | | | |

It was observed that the suspension of salbutamol sulfate in HFA-134a without ethanol demonstrated consistent through life UoDD and delivered the anticipated dose. It was observed that the suspension of salbutamol sulfate in HFA-152a without ethanol demonstrated consistent through life UoDD and delivered the anticipated dose. It was observed that suspension of salbutamol sulfate in HFA-152a with ethanol also demonstrated consistent through life UoDD and delivered the anticipated dose. Therefore, the addition of ethanol in HFA-152a salbutamol sulfate suspensions had no apparent effect on through life UoDD and overall delivery of the anticipated dose

In contrast, it was observed that suspension of salbutamol sulfate in HFO-1234ze(E) without ethanol demonstrated lower than anticipated delivered dose and inconsistent through life UoDD. However, suspensions of salbutamol sulfate in HFO-1234ze(E) with 5% ethanol by weight demonstrated consistent through life UoDD and overall delivery of the anticipated dose.

From this example, it was learned that the addition of 5% ethanol to HFO-1234ze(E) suspensions of salbutamol sulfate improved anticipated delivered dose and consistency of through life UoDD. It was also learned that suspension of salbutamol sulfate in HFA-152a or HFA-134a without ethanol showed anticipated delivered dose and consistent through life UoDD.

### Example 2: Delivered Dose Measurement and End of Life Canister and Valve Deposition of Salbutamol Sulfate Suspensions in HFO-1234Ze(E) with and without Ethanol

Suspensions of micronized salbutamol sulfate were prepared in HFO-1234ze(E). Each suspension included an amount of salbutamol sulfate to provide a nominal dose of 100 μg/actuation (1.91 mg/mL). A first suspension included no ethanol. A second suspension included 0.5% ethanol by weight. A third suspension included 1.0% ethanol by weight. A fourth suspension included 2.0% ethanol by weight. A fifth suspension included 5.0% ethanol by weight.

Devices were prepared by weighing salbutamol sulfate into FEP coated canisters and adding the appropriate quantity of ethanol as required. A 63-μL valve was crimped onto the can and HFO-1234ze(E) was pressure filled into the canister. The units were sonicated for 10 minutes to disperse the salbutamol sulfate.

For each suspension, three units were prepared and each was coupled to a KINDEVA actuator with a 0.4 mm exit orifice diameter. For each suspension, through life uniformity of delivered dose was measured, and a mean value derived. Following end of unit life testing, total drug deposition on the canister and valve was measured. The results of this testing are shown in Table 2.

TABLE 2

| Ethanol (%) | Mean delivered dose | Approximate |
|---|---|---|
| by weight | (μg/actuation) | deposition (μg) |
| 0 | 46 | 2810-2970 |
| 0.5 | 53 | Not measured |
| 1.0 | 62 | 1280-1350 |
| 2.0 | 79 | 483-591 |
| 5.0 | 83 | 288-363 |

It was observed that suspensions of salbutamol sulfate in HFO-1234ze(E) without ethanol demonstrated lower than anticipated mean delivered dose and high levels of salbutamol sulfate deposition within the canister and valve. Increasing ethanol in the composition led to a corresponding increase in mean delivered dose, and decrease in salbutamol sulfate deposition within the canister and valve.

From this example, it was learned that the inclusion of, and increasing amounts of, ethanol in suspensions of salbutamol sulfate in HFO1234ze(E) lead to an increased mean delivered dose and a corresponding decrease in salbutamol sulfate deposition within the canister and valve. It was also learned that the inclusion of between 2% and 5% ethanol by weight in HFO-1234ze(E) suspensions of salbutamol sulfate produced the most significant improvement in mean delivered dose and corresponding decrease in salbutamol sulfate deposition within the canister and valve

### Example 3: Delivered Dose and Particle Size Measurement for Suspensions of Salbutamol Sulfate in HFO-1234Ze(E) without and with Increasing Levels of Ethanol

Suspensions of micronized salbutamol sulfate in HFO-1234ze(E) were prepared. A first suspension included no ethanol. A second suspension included 2% ethanol by weight. A third suspension included 5% ethanol by weight. A fourth suspension included 10% ethanol by weight. A fifth suspension included 15% ethanol by weight. Each suspension included an amount of salbutamol sulfate (1.91 mg/mL) to provide a nominal dose of 100 μg/actuation. Each suspension was filled into FEP coated canisters, crimped with a 63-μL valve and tested with a KINDEVA actuator having an exit orifice diameter of 0.4 mm for testing.

The fine particle mass (FPM), median mass aerodynamic diameter (MMAD), fine particle fraction (FPF), metered dose (ex-valve), and delivered dose (ex-act) were measured for each suspension. Results are shown in Table 3.

TABLE 3

| Ethanol (%) | FPM | MMAD | FPF | Ex-valve | Ex-act |
|---|---|---|---|---|---|
| by weight | (μg/act) | (μm) | (%) | (μg/act) | (μg/act) |
| 0 | 45.3 | 1.9 | 57.5 | 78.9 | 63.8 |
| 2 | 45.9 | 2.0 | 48.5 | 94.5 | 77.7 |
| 5 | 36.8 | 1.9 | 39.1 | 94.4 | 82.7 |
| 10 | 25.4 | 2.0 | 27.6 | 92.1 | 83.9 |
| 15 | 18.5 | 2.0 | 19.6 | 94.6 | 85.8 |

It was observed that suspensions of salbutamol sulfate in HFO-1234ze(E) including increasing levels of ethanol showed a corresponding decrease in FPF and FPM.

From this example, it was learned that the inclusion of, and increasing amounts of, ethanol in suspensions of salbutamol sulfate in HFO-1234ze(E) lead to a decrease in FPF and FPM.

It was also learned that at ethanol levels less than 2% by weight, the ex-actuator delivered dose was lower than anticipated even though the corresponding FPM and FPF values were high. It was notable that with ethanol levels of 2% by weight and greater resulted in an appropriate and anticipated ex-actuator delivered dose. However, increasing ethanol levels beyond 5% by weight, resulted in no further benefit in mean ex-actuator delivered dose, whilst resulting in a significant and undesirable reduction in FPM and FPF. Therefore, it was learned that the most beneficial level of ethanol in the composition to provide maximum benefit to both delivered dose and FPM/FPF is between 2% and 5% ethanol by weight in HFO-1234ze(E) suspensions of salbutamol sulfate.

### Example 4: Delivered Dose of Salbutamol Sulfate in HFO-1234Ze(E) with 5.0% or 15% Ethanol

Suspensions of micronized salbutamol sulfate in HFO-1234ze(E) were prepared. A first suspension of salbutamol sulfate and 5.0% ethanol by weight was prepared in HFO-1234ze(E). A second suspension of salbutamol sulfate and 15% ethanol by weight was prepared in HFO-1234ze(E). The concentration of salbutamol sulfate in each formulation was 1.91 mg/mL. Each suspension was filled into FEP coated canisters, crimped with a valve, and coupled to a 63-μL APTAR actuator for testing.

Fine particle mass and delivered dose were measured for each suspension. The results of these tests as a mean value of triplicate measurements are shown in Table 4.

TABLE 4

| Ethanol | Delivered | Fine particle |
|---|---|---|
| (%) | dose (μg/act) | mass (%) |
| 5.0 | 87 | 38 |
| 15 | 92 | 20 |

It was observed that the suspension of salbutamol sulfate in HFO-1234ze(E) with 15% ethanol demonstrated a slightly higher delivered dose and lower fine particle mass. It was observed that each suspension had a similar delivered dose. From this example, it was learned that a suspension of salbutamol sulfate in HFO-1234ze(E) and 5% ethanol had higher fine particle mass than a comparable suspension with 15% ethanol.

### Example 5: Physical and Chemical Stability of Salbutamol Sulfate in HFO-1234Ze(E) Suspensions with 5% Ethanol Over 26 Weeks at 40° C. and 75% Relative Humidity and 25° C. and 60% Relative Humidity

Suspensions of micronized salbutamol sulfate in HFO-1234ze(E) with 5% ethanol by weight were prepared. Each suspension included an amount of salbutamol sulfate (1.91 mg/mL) to provide a nominal dose of 100 μg/actuation. Each suspension was filled into FEP coated canisters, crimped with either a 63-μL APTAR or KINDEVA metering valve and tested with a KINDEVA actuator having an exit orifice diameter of 0.4 mm.

A first group of suspensions with each canister/valve combination were stored at 40° C. and 75% relative humidity in the valve down orientation. FPM through life UoDD, salbutamol sulfate content and impurities for each canister/valve combination were measured at initial, 6, 13, and 26 weeks post-preparation.

It was observed that, for each canister/valve combination, the FPM, uniformity of delivered dose and salbutamol sulfate content remained consistent over the duration of 26 weeks stability storage. It was also observed that for each canister/valve combination, minimal impurities were detected over the duration of 26 weeks stability storage.

A second group of suspensions with each canister/valve combination were stored at 25° C. and 60% relative humidity in the valve down orientation. FPM, through life uniformity of delivered dose, salbutamol sulfate content and impurities content for each canister/valve combination were measured at initial and 26 weeks post-preparation.

It was observed that, for each canister/valve combination, the FPM, through life uniformity of delivered dose, salbutamol sulfate content remained consistent and minimal impurities were observed over the duration of 26 weeks stability storage duration post-preparation.

From this example, it was learned that a suspension of salbutamol sulfate in HFO-1234ze(E) with 5% ethanol by weight was physically and chemically stable, as demonstrated by consistent FPM, through life uniformity of delivered dose, salbutamol sulfate content and minimal impurities over the duration of 26 weeks when stored at either 40° C. and 75% relative humidity or 25° C. and 60% relative humidity. The mean results demonstrating the above learnings are presented in Table 5.

TABLE 5

| | | | 6 Weeks | 13 Weeks | 26 Weeks | |
|---|---|---|---|---|---|---|
| | | 0 Weeks | 40° C./ | 40° C./ | 40° C./ | 25° C./ |
| Test | Valve | Ambient | 75% RH | 75% RH | 75% RH | 60% RH |
| Mean Salbutamol | APTAR | 1.6 | 1.5 | 1.6 | 1.6 | 1.6 |
| Content (mg/mL) | KINDEVA | 1.6 | 1.6 | 1.6 | 1.6 | 1.6 |
| Mean Fine | APTAR | 38.1 | 37.3 | 41.0 | 40.6 | 39.8 |
| Particle Mass | KINDEVA | 32.9 | 33.1 | 34.1 | 36.3 | 34.2 |
| (μg/actuation) | | | | | | |
| Mean Through | APTAR | 84.5 | 84.2 | 86.6 | 80.1 | 77.4 |
| Life Delivered Dose | KINDEVA | 82.1 | 80.1 | 82.0 | 78.9 | 75.5 |
| (μg/actuation) | | | | | | |
| Maximum Total | APTAR | 0.06 | 0.00 | 0.06 | 0.10 | 0.00 |
| Impurities (% w/w) | KINDEVA | 0.06 | 0.00 | 0.09 | 0.12 | 0.00 |

### Comparative Example 6: Delivery of Fluticasone Propionate Suspension in HFO-1234Ze(E) without Ethanol

This Example presents data describing suspensions of micronized fluticasone propionate without ethanol or other excipients. These data are presented as a comparative example, motivating the inclusion of ethanol in the formulations of the present disclosure.

A suspension of micronized fluticasone propionate was prepared in HFO-1234ze(E) with no additional excipients, including ethanol. Each suspension included an amount of fluticasone propionate (2 mg/mL) to provide a nominal dose of 100 μg/actuation. Each suspension was filled into FEP coated canisters and crimped with a metering valve and tested with a KINDEVA actuator.

With a 0.5 mm exit orifice diameter actuator, the through life mean delivered dose and aerodynamic particle size distribution (APSD) were tested.

It was observed that a lower than anticipated mean delivered dose of 71 μg/actuation was achieved.

When tested with KINDEVA actuators with exit orifice diameters of 0.3 mm, 0.22 mm, and 0.18 mm using a Fast Screening Impactor (FSI), it was observed that corresponding FPF of 35%, 43%, and 50% were achieved.

From these examples, it was learned that an excipient-free suspension of fluticasone propionate in HFO-1234ze(E) delivered using a 0.5 mm exit orifice diameter actuator delivered a lower than desired dose. It was also learned that reducing actuator exit orifice diameter to 0.3 mm, 0.22 mm, and 0.18 mm progressively improved the fine particle fraction of excipient free suspensions of fluticasone propionate in HFO-1234ze(E).

### Example 7: Delivered Dose Measurement and End of Life Canister and Valve Deposition of Fluticasone Propionate Suspensions in HFO-1234Ze(E) with and without Added Ethanol

Suspensions of micronized fluticasone propionate were prepared in HFO-1234ze(E). The nominal concentration of fluticasone propionate in each suspension was 2 mg/mL. A first suspension included 0% ethanol. A second suspension included 0.5% ethanol by weight. A third suspension included 1.0% ethanol by weight. A fourth suspension included 2.0% ethanol by weight.

Each suspension was filled into an FEP-coated canister and fitted with a 50-μL valve. The suspensions were sonicated for 10 minutes to disperse the fluticasone propionate. The suspensions were tested using a KINDEVA actuator with a 0.5-mm exit orifice diameter.

For each suspension, through life UoDD was measured and a mean value derived. Following end of unit life testing, total drug deposition on the canister and valve was measured. The results of this testing are shown in Table 6.

It was observed that suspensions of fluticasone propionate in HFO-1234ze(E) without ethanol demonstrated significantly lower than anticipated mean delivered dose and high levels of fluticasone propionate deposition within the canister and valve. Increasing ethanol in the suspension formulation led to a corresponding increase in mean delivered dose, and decrease in fluticasone propionate deposition within the canister and valve.

From this example, it was learned that the inclusion of, and increasing amounts of, ethanol in suspensions of fluticasone propionate in HFO-1234ze(E) lead to an increased mean delivered dose and a corresponding decrease in fluticasone propionate deposition within the canister and valve. It was also learned that the inclusion of between 1% to 2% ethanol by weight in HFO-1234ze(E) suspensions of fluticasone propionate produced the most significant improvement in mean delivered dose and corresponding decrease in fluticasone propionate deposition within the canister and valve. Therefore, it was learned that the most beneficial level of ethanol in the composition to provide maximum benefit to both delivered dose and deposition on the canister and valve is between 1% and 2% ethanol by weight in HFO-1234ze(E) suspensions of fluticasone propionate.

TABLE 6

| Ethanol (%) | Mean delivered dose | Approximate |
|---|---|---|
| by weight | (μg/actuation) | deposition (μg) |
| 0 | 38 | 2200-4200 |
| 0.5 | 63 | 1740-1870 |
| 1.0 | 84 | 1200-1220 |
| 2.0 | 88 | 800-900 |

### Example 8: Aerodynamic Particle Size Measurement of Fluticasone Propionate Suspensions in HFO-1234Ze(E) with and without Added Ethanol

Suspensions of micronized fluticasone propionate were prepared in HFO-1234ze(E). The nominal concentration of fluticasone propionate in each suspension was 2 mg/mL. A first suspension included 0% ethanol. A second suspension included 2% ethanol by weight. A third suspension included 5% ethanol by weight. A fourth suspension included 10% ethanol by weight.

Each suspension was filled into an FEP-coated canister and fitted with a 50-μL valve. The suspensions were sonicated for 10 minutes to disperse the fluticasone propionate. The suspensions were tested using a KINDEVA actuator with a 0.5 mm exit orifice diameter.

For each suspension, FPM, median mass aerodynamic diameter (MMAD), FPF, metered dose (ex-valve), and delivered dose (ex-actuator) were measured. The mean results of this testing are shown in Table 7.

TABLE 7

| | | | | Ex-valve | Ex-actuator |
|---|---|---|---|---|---|
| | FPM | | | dose | dose |
| Ethanol (%) | (μg/ | MMAD | FPF | (μg/ | (μg/ |
| by weight | actuation) | (μm) | (%) | actuation) | actuation) |
| 0% | 5.3 | 5.0 | 60.6 | 11.4 | 8.8 |
| 2% | 30.0 | 5.4 | 43.8 | 77.0 | 68.5 |
| 5% | 27.9 | 2.4 | 28.9 | 103.7 | 96.6 |
| 10% | 18.7 | 2.1 | 19.6 | 101.9 | 95.2 |

It was observed in this example that suspensions of fluticasone propionate in HFO-1234ze(E) without ethanol demonstrated significantly lower than anticipated ex-actuator dose and FPM Additionally, increasing ethanol from 2% to 10% by weight in the suspension formulation led to a corresponding increase in ex-actuator dose, and corresponding reduction in FPM, FPF and MMAD.

It was learned that without ethanol, the ex-actuator delivered dose was significantly lower than anticipated even though the corresponding FPF values were high.

It was notable that ethanol levels of 2% by weight and greater resulted in an appropriate and anticipated ex-actuator delivered dose. However, increasing ethanol levels beyond 5% by weight, resulted in no further benefit in mean ex-actuator delivered dose, whilst resulting in a significant and undesirable reduction in FPM and FPF. Therefore, it was learned that the most beneficial level of ethanol in the composition to provide maximum benefit to both delivered dose and FPM/FPF is between 2% and 5% ethanol by weight in HFO-1234ze(E) suspensions of fluticasone propionate.

### Example 9: Solubility of Suspended Particles Including Fluticasone Propionate in HFO-1234Ze(E)

The saturated solubility of micronized fluticasone propionate when suspended in HFO-1234ze(E) with ethanol ranging from 0% to 10% by weight and no additional excipients was measured. The solubility of fluticasone propionate in each suspension was measured by content assay following filtration of the excess non-dissolved drug. Data from this testing is shown in Table 8.

It was observed that in a suspension including no ethanol, fluticasone propionate demonstrated extremely low solubility in HFO-1234ze(E). Solubility in the suspension formulation was found to increase as ethanol content increased. Elevated levels of solubility at 5% ethanol or greater could give rise to undesirable fluticasone propionate particle growth within the suspension formulation over time as descried herein.

TABLE 8

| Ethanol (%) | Mean fluticasone propionate |
|---|---|
| by weight | solubility (mg/mL) |
| 0% | 0.0032 |
| 1% | 0.0124 |
| 2% | 0.0303 |
| 5% | 0.1349 |
| 10% | 0.3675 |

From this example, it was learned that the solubility of fluticasone propionate in HFO-1234ze(E) suspension formulations increased as the concentration of ethanol increased. This solubility of fluticasone propionate was more pronounced in HFO-1234ze(E) fluticasone propionate suspension formulations containing 5% or 10% ethanol by weight.

### Example 10: Delivered Dose Measurement and End of Life Canister and Valve Deposition of Mometasone Furoate Suspensions in HFO-1234Ze(E) with and without Added Ethanol

Suspensions of micronized mometasone furoate were prepared in HFO-1234ze(E). The nominal concentration of mometasone furoate in each suspension was 2 mg/mL. A first suspension included 0% ethanol. A second suspension included 0.5% ethanol by weight. A third suspension included 1.0% ethanol by weight. A fourth suspension included 2.0% ethanol by weight.

Each suspension was filled into an FEP-coated canister and fitted with a 50-μL valve. The suspensions were sonicated for 10 minutes to disperse the mometasone furoate. The suspensions were tested using a KINDEVA actuator with a 0.5 mm exit orifice diameter.

For each suspension, through life uniformity of delivered dose was measured, and a mean value derived. For the suspensions containing 0% and 1% ethanol by weight, following end of unit life testing, total drug deposition on the canister and valve was measured. The results of this testing are shown in Table 9.

TABLE 9

| | Mean dose | | Mean mometasone |
|---|---|---|---|
| Ethanol (%) | delivered | Deposition | solubility |
| by weight | (μg/act) | (μg) | (mg/mL) |
| 0 | 78 | 1534-1901 | 0.0008 |
| 0.5 | 77 | Not measured | Not measured |
| 1 | 88 | 483-601 | 0.0076 |
| 2 | 92 | Not measured | Not measured |

It was observed that suspensions of mometasone furoate in HFO-1234ze(E) without ethanol demonstrated lower than anticipated mean delivered dose and high levels of mometasone furoate deposition within the canister and valve. Increasing ethanol in the suspension formulation above 0.5% by weight led to a corresponding increase in mean delivered dose. It was also observed that 1% ethanol by weight in the suspension formulation led to a decrease in mometasone furoate deposition within the canister and valve when compared to suspensions of mometasone furoate in HFO-1234ze(E) without ethanol.

From this example, it was learned that the inclusion of ethanol at 1% and 2% by weight in HFO-1234ze(E) suspensions of mometasone furoate lead to an increased delivered dose and was closer to the anticipated mean value. It was also learned that the inclusion of ethanol at 1% by weight in HFO-1234ze(E) suspensions of mometasone furoate led to a decrease in mometasone furoate deposition within the canister and valve when compared to suspensions of mometasone furoate in HFO-1234ze(E) without ethanol. Therefore, it was learned that the most beneficial level of ethanol in the composition to provide maximum benefit to both delivered dose and deposition on the canister and valve is between 1% and 2% ethanol by weight in HFO-1234ze(E) suspensions of mometasone furoate. At 1% by weight ethanol, there was a marginal increase of solubility of mometasone relative to no ethanol.

### Example 11: Suspensions of Mometasone Furoate in HFO-1234Ze(E)

Suspensions of micronized mometasone furoate were prepared in HFO-1234ze(E). The nominal concentration of mometasone furoate was 2 mg/mL. A first suspension included 0% ethanol. A second suspension included 0.5% ethanol by weight. A third suspension included 1% ethanol by weight. A fourth suspension included 2% ethanol by weight.

Devices were prepared by weighing mometasone furoate in FEP coated canisters fitted with a 50-μL valve, a Mk6s actuator, and dose counter. The valve was crimped on, and the propellant was pressure filled through the valve. The units were sonicated for 10 minutes to fully disperse the mometasone furoate. Through life dosing was tested for each suspension 3 units with the Mk6s actuator having 0.5-mm exit orifice and 0.8-mm jet length. Saturated solubility was also measured for each suspension. The results are shown in Example 10.

TABLE 10

| | Mean dose | Saturated | |
|---|---|---|---|
| Ethanol | delivered | solubility | Deposition |
| (%) | (μg/act) | (mg/mL) | (μg) |
| 0 | 78 | 0.0008 | 1580 |
| 0.5 | 77 | Not measured | Not measured |
| 1 | 88 | Not measured | 2030 |
| 2 | 92 | 0.0076 | Not measured |

It was observed that inclusion of ethanol in mometasone furoate suspension formulations in HFO-1234ze(E) resulted in improved mean delivered dose and lower valve and canister mometasone deposition relative to an ethanol free formulation.

### Example 12: Delivered Dose Measurement of Budesonide Suspensions in HFO-1234Ze(E) with and without Ethanol

Suspensions of micronized budesonide with a mean geometric d50 particle size of 2.2 μm, were prepared in HFO-1234ze(E). For all suspensions, the concentration of budesonide (2 mg/mL) was selected to deliver a nominal dose of 100 μg/actuation from a 50-μL valve. A first suspension included 0% ethanol. A second suspension included 1.0% ethanol by weight. A third suspension included 2.0% ethanol by weight. Each suspension was high shear mixed for 15 minutes to disperse the suspended budesonide, prior to being filled into FEP-coated canisters and coupled to a 50-μL valve.

Through unit life (TUL) uniformity of delivered dose was measured in triplicate for preparations of each suspension formulation using a KINDEVA actuator with an exit orifice diameter of 0.4 mm. The results are presented in Table 11.

TABLE 11

| | Start | Middle | End | | Standard |
|---|---|---|---|---|---|
| | of Unit | of Unit | of Unit | Mean | Deviation |
| | Life Mean | Life Mean | life Mean | TUL | of TUL |
| | Delivered | Delivered | Delivered | Delivered | Delivered |
| Ethanol | Dose | Dose | Dose | Dose | Dose |
| (%) by | (μg/ | (μg/ | (μg/ | (μg/ | (μg/ |
| weight | actuation) | actuation) | actuation) | actuation) | actuation) |
| 0 | 70.8 | 105.4 | 98.3 | 91.5 | 28.4 |
| 1.0 | 83.2 | 59.3 | 67.8 | 70.1 | 16.9 |
| 2.0 | 85.7 | 78.8 | 75.5 | 80.0 | 9.5 |

It was observed that the inclusion of either 1.0% or 2.0% ethanol by weight improved through unit life delivered dose consistency as demonstrated by a reduction in the standard deviation of the mean through unit delivered dose. The budesonide suspension including 2.0% ethanol by weight demonstrated less through life delivered dose variability than a suspension without ethanol. From this example, it was learned that budesonide suspensions in HFO-1234ze(E) benefited from the inclusion of 1.0% and 2.0% ethanol by weight as variability of through unit life delivered dose was reduced relative to suspensions without ethanol.

The embodiments described above and illustrated in the figures are presented by way of example only and are not intended as a limitation upon the concepts and principles of the present disclosure. As such, it will be appreciated by one having ordinary skill in the art that various changes in the elements and their configuration and arrangement are possible without departing from the spirit and scope of the present disclosure. All references and publications cited herein are expressly incorporated herein by reference in their entirety into this disclosure. Various features and aspects of the present disclosure are set forth in the following claims.

1. A metered dose inhaler comprising:
a metering valve;
a canister; and
an actuator comprising an actuator nozzle;
wherein the canister comprises a formulation, the formulation comprising:
greater than 70% by weight of propellant HFO-1234ze(E);
ethanol; and
at least one active pharmaceutical ingredient suspended in the formulation to form a suspension.

2.-20. (canceled)

21. A metered dose inhaler comprising:
a metering valve;
a canister; and
an actuator comprising an actuator nozzle;
wherein the canister comprises a formulation, the formulation comprising:
greater than 98% by weight of propellant HFO-1234ze(E);
polyethylene glycol (PEG) at a concentration of between 0.01 wt % and 1 wt %; and
at least one active pharmaceutical ingredient (API) suspended in the formulation to form a suspension, the API comprising:
a formoterol salt or solvate; and
a corticosteroid;
wherein the formulation does not include ethanol.

22. The inhaler of claim 21, wherein the corticosteroid comprises budesonide or mometasone.

23. The inhaler of claim 21, wherein the formulation comprises the active pharmaceutical ingredient to deliver an amount greater than 0.001 mg/actuation.

24. The inhaler of claim 23, wherein the formulation comprises the active pharmaceutical ingredient to deliver an amount less than 0.5 mg/actuation.

25. The inhaler of claim 21, wherein HFO-1234ze(E) is the sole propellant.

26. The inhaler of claim 21, wherein the formulation comprises between 0.01% and 0.1% of PEG.

27. The inhaler of claim 21, wherein the formulation comprises PEG 1000 or PEG 300.

28. The inhaler of claim 21, wherein the formulation does not include polyvinylpyrrolidone.

29. The inhaler of claim 21, wherein the metering valve has a size of 25 μL, 50 μL, or 63 μL.

30. The inhaler of claim 21, wherein the actuator nozzle comprises an exit orifice effective diameter of 0.12 mm to 0.5 mm.

31. The inhaler of claim 21, wherein the formoterol comprises formoterol fumarate.

32. A metered dose inhaler comprising:
a metering valve;
a canister; and
an actuator comprising an actuator nozzle;
wherein the canister comprises a formulation, the formulation comprising:
greater than 98% by weight of the propellant HFO-1234ze(E);
PEG 1000 at a concentration of between 0.01% and 1%; and
an API suspended in the formulation to form a suspension, the API comprising formoterol fumarate and budesonide.

33. The inhaler of claim 32, wherein HFO-1234ze(E) is the sole propellant.

34. The inhaler of claim 32, wherein the formulation comprises 0.05 wt % to 1.0 wt % of budesonide.

35. The inhaler of claim 32, wherein the formulation includes budesonide in an amount of 50 μg/actuation to 200 μg/actuation and wherein the formulation includes formoterol fumarate at in an amount of 1 μg/actuation to 10 μg/actuation.

36. The inhaler of claim 32, wherein the formulation comprises between 0.01% and 0.1% of PEG.

37. A metered dose inhaler comprising:
a metering valve;
a canister; and
an actuator comprising an actuator nozzle;
wherein the canister comprises a formulation, the formulation comprising:
greater than 98% by weight of the propellant HFO-1234ze(E);
PEG 1000 at a concentration of between 0.01 wt % and 1 wt %; and
an API suspended in the formulation to form a suspension, the API comprising formoterol fumarate and mometasone furoate.

38. The inhaler of claim 37, wherein HFO-1234ze(E) is the sole propellant.

39. The inhaler of claim 37, wherein the formulation comprises 0.05 wt % and 1.0 wt % of mometasone furoate.

40. The inhaler of claim 37, wherein the formulation includes mometasone furoate in an amount of 50 μg/actuation to 200 μg/actuation and wherein the formulation includes formoterol fumarate at in an amount of 1 μg/actuation to 10 μg/actuation.
